(19) United States
(12) Patent Application Publication
(10) Pub. No.: US 2025/0281142 A1
(43) Pub. Date: Sep. 11, 2025

(54) X-RAY CT APPARATUS

(71) Applicant: FUJIFILM Corporation, Tokyo (JP)

(72) Inventor: Taiga GOTO, Tokyo (JP)

(73) Assignee: FUJIFILM Corporation, Tokyo (JP)

(21) Appl. No.: 19/071,650

(22) Filed: Mar. 5, 2025

(30) Foreign Application Priority Data
Mar. 7, 2024 (JP) 2024-034793

(51) Int. Cl. A61B 6/00; A61B 6/03; G16H 40/63

(52) U.S. Cl. CPC A61B 6/542; A61B 6/03; A61B 6/4452; A61B 6/5205; G16H 40/63

(57) ABSTRACT

Provided is an X-ray CT apparatus capable of suppressing a decrease in image quality of a tomographic image due to X-ray attenuation in a head holder or a top plate. The X-ray CT apparatus includes an X-ray source that irradiates an X-ray to a subject, an X-ray detector that detects the X-ray transmitted through the subject, a rotating plate that causes the X-ray source and the X-ray detector to rotate around the subject, an image generation section that generates a tomographic image of the subject based on a detection signal of the X-ray detector, and a controller that controls each section, in which the controller changes an irradiation condition of the X-ray irradiated from the X-ray source while the rotating plate rotates, such that an irradiation dose from a side on which a head holder or a top plate is disposed is increased in a range of a projection angle at which the X-ray passes through the head holder or the top plate.

### CLAIM OF PRIORITY

The present application claims priority from Japanese Patent Application JP 2024-034793 filed on Mar. 7, 2024, the content of which is hereby incorporated by reference into this application.

### BACKGROUND OF THE INVENTION

### 1. Field of the Invention

The present invention relates to an X-ray CT apparatus that captures a tomographic image of a subject, and particularly relates to a technique of reducing exposure of a subject.

### 2. Description of the Related Art

An X-ray CT apparatus uses projection data from multiple directions that is obtained by rotating, around a subject, an X-ray source that irradiates an X-ray to the subject and an X-ray detector that detects the X-ray transmitted through the subject to generate a tomographic image of the subject. The generated tomographic image depicts a shape of an organ within the subject and is used for image diagnosis.

Image quality of the tomographic image is improved as a dose of the X-ray irradiated to the subject is higher, but an exposure dose to the subject increases. Since the increase in the exposure dose has a bad influence on the subject, it is important to suppress an irradiation dose, which is an X-ray dose to be irradiated, particularly to a site positioned on a body surface and having high radiation sensitivity, such as an eye or a mammary gland.

U.S. Pat. No. 9,867,587A discloses that an irradiation dose in a case where an X-ray source that rotates around a subject is positioned on a front side of the subject, which is a side of a site having high radiation sensitivity, is reduced as compared with the irradiation dose in a case where the X-ray source is positioned on a rear side of the subject.

### SUMMARY OF THE INVENTION

However, in U.S. Pat. No. 9,867,587A, there is insufficient consideration for X-ray attenuation by a head holder on which the head part of the subject is placed and a top plate on which the subject is placed. The X-ray attenuation by the head holder or the top plate reduces the X-ray dose detected by the X-ray detector, which reduces the image quality of the tomographic image. In addition, in a case in which the irradiation dose is increased in order to compensate for the X-ray attenuation by the head holder or the top plate, an exposure dose to the subject increases.

Therefore, an object of the present invention is to provide an X-ray CT apparatus capable of suppressing a decrease in image quality of a tomographic image due to X-ray attenuation in a head holder or a top plate.

In order to achieve the above object, according to the present invention, there is provided an X-ray CT apparatus comprising: an X-ray source that irradiates an X-ray to a subject; an X-ray detector that detects the X-ray transmitted through the subject; a rotating plate that causes the X-ray source and the X-ray detector to rotate around the subject; an image generation section that generates a tomographic image of the subject based on a detection signal of the X-ray detector; and a controller that controls each section, in which the controller changes an irradiation condition of the X-ray irradiated from the X-ray source while the rotating plate rotates, such that an irradiation dose from a side on which a head holder or a top plate is disposed is increased in a range of a projection angle at which the X-ray passes through the head holder or the top plate.

According to the present invention, it is possible to provide an X-ray CT apparatus capable of suppressing a decrease in image quality of a tomographic image due to X-ray attenuation in a head holder or a top plate.

### BRIEF DESCRIPTION OF THE DRAWINGS

FIG. 1 is a diagram showing an example of an overall configuration of an X-ray CT apparatus of Example 1.

FIG. 2 is a diagram for describing modulation of an irradiation dose.

FIG. 3 is a diagram showing an example of a flow of processing of Example 1.

FIG. 4 is a diagram showing an example of a screen used for selection of a head holder.

FIG. 5 is a diagram showing an example of a change in X-ray irradiation condition.

FIG. 6 is a diagram showing another example of the change of the X-ray irradiation condition.

FIG. 7 is a diagram showing an example of an overall configuration of an X-ray CT apparatus of Example 2.

FIG. 8 is a diagram showing an example of a flow of processing of Example 2.

FIG. 9 is a diagram showing an example of a camera image.

### DESCRIPTION OF THE PREFERRED EMBODIMENTS

Hereinafter, examples of an X-ray computed tomography (CT) apparatus according to the embodiments of the present invention will be described with reference to accompanying drawings. The X-ray CT apparatus generates a tomographic image as a medical image used for diagnosis of a subject or the like. In the following description and the accompanying drawings, components having the same function are designated by the same reference numerals, and duplicate description thereof will be omitted.

### Example 1

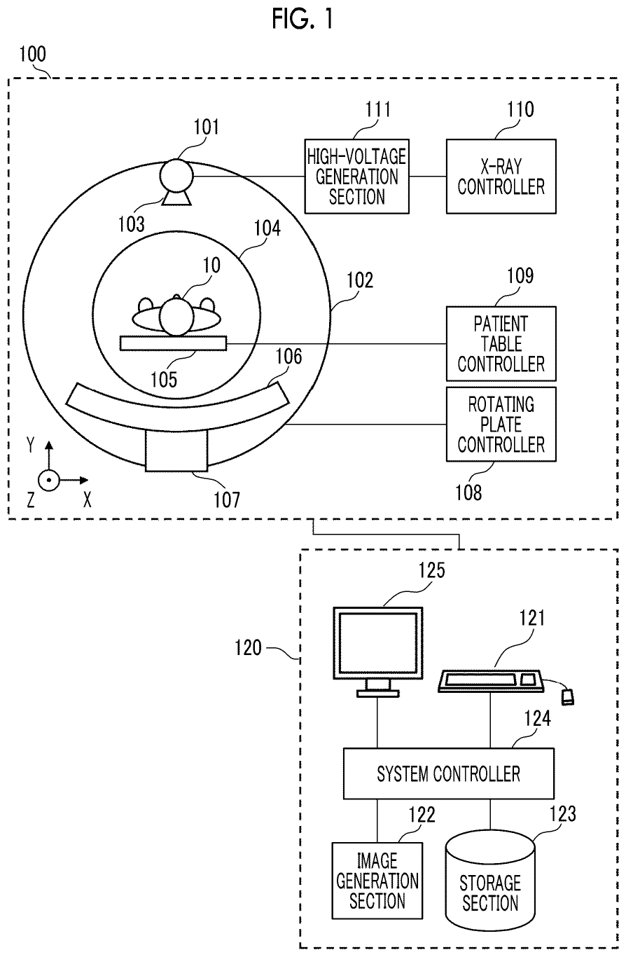

An overall configuration of the X-ray CT apparatus of Example 1 will be described with reference to FIG. 1. The X-ray CT apparatus comprises a scan gantry unit 100 and an operation unit 120. The scan gantry unit 100 is installed in an imaging room surrounded by a shielding material that blocks an X-ray, and the operation unit 120 is installed in an operation room located outside the imaging room.

The scan gantry unit 100 comprises an X-ray source 101, a rotating plate 102, a collimator 103, an X-ray detector 106, a data collection section 107, an patient table 105, a rotating plate controller 108, an patient table controller 109, an X-ray controller 110, and a high-voltage generation section 111. The X-ray source 101 is a device that irradiates the X-rays to a subject 10 placed on the patient table 105 and is, for example, an X-ray tube device. The collimator 103 is a device that restricts an irradiation range of the X-ray. The rotating plate 102 is provided with an gantry aperture 104 through which the subject 10 placed on the patient table 105 enters, and is also equipped with the X-ray source 101 and the X-ray detector 106 and rotates the X-ray source 101 and the X-ray detector 106 around the subject 10. The rotation axis of the rotating plate 102 is referred to as a Z-axis, a horizontal plane is referred to as a ZX plane, and a vertical axis is referred to as a Y-axis.

The X-ray detector 106 is a device that is disposed to face the X-ray source 101 and comprises a plurality of detection elements that detect the X-ray to acquire a spatial distribution of the X-ray. The detection elements of the X-ray detector 106 are arranged two-dimensionally in a rotation direction and a rotation axis direction of the rotating plate 102. The data collection section 107 is a device that collects the spatial distribution of the X-ray acquired by the X-ray detector 106 as digital data.

The rotating plate controller 108 is a device that controls rotation and inclination of the rotating plate 102. The patient table controller 109 is a device that controls up, down, front, back, left, and right movements of the patient table 105. The high-voltage generation section 111 is a power source that generates a tube voltage, which is a voltage applied to the X-ray source 101, and a tube current, which is a current supplied to the X-ray source 101. The X-ray controller 110 is a device that controls an output of the high-voltage generation section 111. The rotating plate controller 108, the patient table controller 109, and the X-ray controller 110 are, for example, a micro-processing unit (MPU) or the like.

The operation unit 120 comprises an input section 121, an image generation section 122, a display section 125, a storage section 123, and a system controller 124. The input section 121 is a device that is used to input examination data such as a name of the subject 10, an examination date and time, and an imaging condition, and is, for example, a keyboard, a pointing device, a touch panel, or the like. The image generation section 122 is a device that generates the tomographic image by using the digital data collected by the data collection section 107, and is, for example, an MPU, a graphics processing unit (GPU), or the like. The display section 125 is a device that displays the tomographic image or the like generated by the image generation section 122, and is, for example, a liquid crystal display, a touch panel, or the like. The storage section 123 is a device that stores the digital data collected by the data collection section 107, the tomographic image generated by the image generation section 122, a program to be executed by the system controller 124, data to be used by the program, and the like, and is, for example, a hard disk drive (HDD), a solid state drive (SSD), or the like. The system controller 124 is a device that controls each unit such as the rotating plate controller 108, the patient table controller 109, and the X-ray controller 110, and is, for example, a central processing unit (CPU).

With the generation of the tube voltage and the tube current by the high-voltage generation section 111 based on the imaging condition set via the input section 121, the X-ray according to the imaging condition is irradiated from the X-ray source 101 to the subject 10. The X-ray detector 106 detects the X-rays irradiated from the X-ray source 101 and transmitted through the subject 10 with a large number of detection elements to acquire the spatial distribution of the transmitted X-rays. The rotating plate 102 is controlled by the rotating plate controller 108 to rotate based on the imaging condition input through the input section 121, particularly a rotation speed or the like. The patient table 105 is controlled by the patient table controller 109 and moves relative to the rotating plate 102.

With repetition of the irradiation of the X-ray by the X-ray source 101 and the detection of the X-ray by the X-ray detector 106 together with the rotation of the rotating plate 102, projection data, which is an X-ray projection image of the subject 10, is measured at various projection angles. In the projection data, a view representing each projection angle is associated with a channel (ch) number and a column number which are detection element numbers of the X-ray detector 106. The measured projection data is transmitted to the image generation section 122. The image generation section 122 performs back-projection processing on a plurality of pieces of projection data to generate the tomographic image. The generated tomographic image is displayed on the display section 125 or stored in the storage section 123 as the medical image.

In the X-ray CT apparatus, image quality of the generated tomographic image is improved as an irradiation dose, which is an X-ray dose irradiated to the subject 10, is higher, but an exposure dose to the subject 10 increases. Since the increase in the exposure dose has a bad influence on the subject 10, the irradiation dose is required to be suppressed. In particular, in order to reduce the exposure dose to a site positioned on a body surface and having high radiation sensitivity, such as an eye or a mammary gland, the irradiation dose to the subject 10 may be modulated during the rotation of the rotating plate 102.

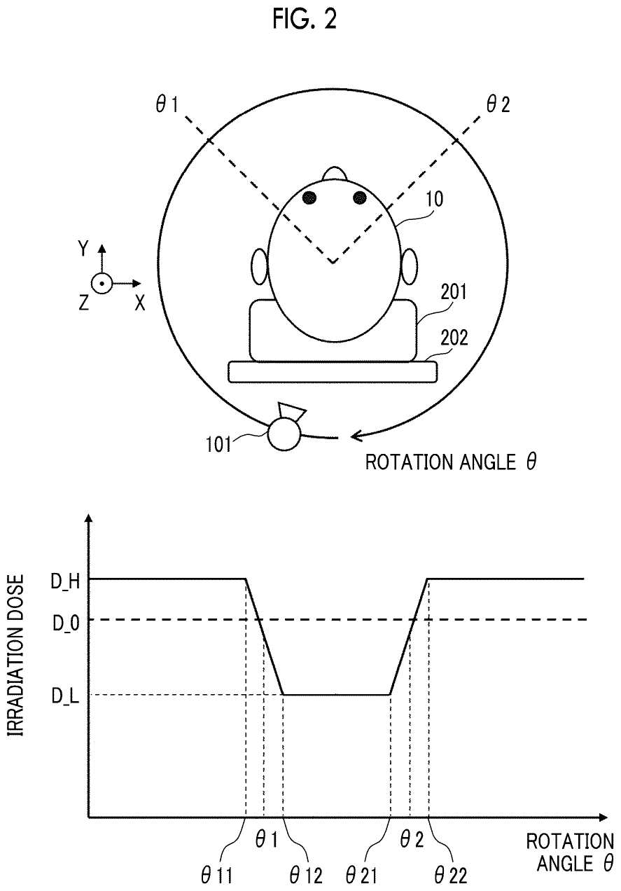

The irradiation dose modulated during the rotation of the rotating plate 102 will be described with reference to FIG. 2. An upper part of FIG. 2 shows the X-ray source 101 that rotates around a head part of the subject 10. The imaging site of the subject 10 is not limited to the head part. In a case where a rotation angle θ of the X-ray source 101 is in a range from θ1 to θ2, the X-ray is irradiated to the eye, which is the site having high radiation sensitivity. Thus, in order to reduce the exposure dose to the eye, the irradiation dose is modulated in accordance with the rotation angle θ. The rotation angle θ corresponds to the projection angle. In addition, in FIG. 2, the negative direction of the Y axis is set to θ=0.

A lower part of FIG. 2 shows an example of a modulation pattern of the irradiation dose by a solid line. The vertical axis is the irradiation dose, the horizontal axis is the rotation angle θ , and the irradiation dose before modulation is indicated by a dotted line for comparison. Since the X-ray source 101 rotates at a constant speed, the rotation angle θ is proportional to time. In the modulation pattern shown in FIG. 2, the dose amount is set to D_L, which is less than the irradiation dose D_0 before modulation, at θ12<θ<θ21 such that the exposure dose at θ1<θ<θ2 is reduced. In order to compensate for the reduction in the irradiation dose in θ12<θ<θ21, the dose amount is set to D_H, which is larger than the irradiation dose D_0 before modulation in θ<θ11 and θ22<θ. Further, the irradiation dose changes from D_H to D_L in θ11<θ<θ12, and the irradiation dose changes from D_L to D_H in θ21<θ<θ22.

By the way, the head holder 201 on which the head part of the subject 10 is placed or the top plate 202 on which the subject 10 is placed attenuates the X-rays irradiated to the subject 10. The X-ray attenuation by the head holder 201 or the top plate 202 increases noise in the tomographic image and decreases the image quality in order to reduce the X-ray dose detected by the X-ray detector 106. Therefore, in Example 1, the irradiation dose is adjusted to compensate for the X-ray attenuation by the head holder 201 or the top plate 202. Specifically, in a range of projection angle at which the X-ray passes through the head holder 201 or the top plate 202, the irradiation dose from the side on which the head holder 201 or the top plate 202 is disposed is increased to suppress a decrease in the image quality of the tomographic image.

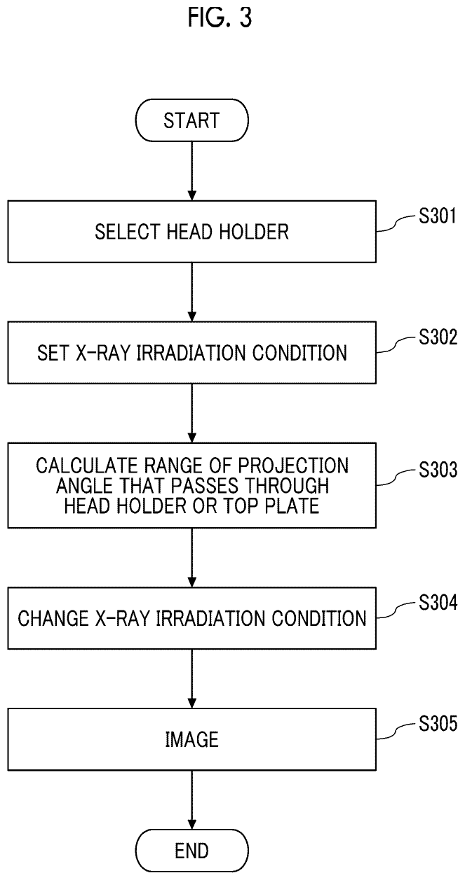

An example of a flow of processing of Example 1 will be described step by step with reference to FIG. 3.

### S301

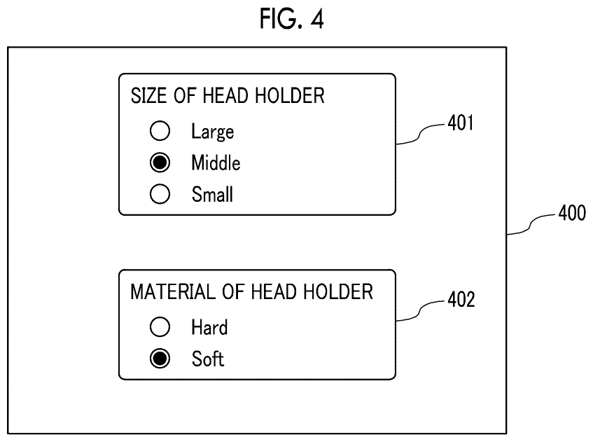

The operator selects the type of the head holder 201 disposed below the head part of the subject 10 via the operation unit 120. For the selection of the type of the head holder 201, for example, a head holder selection screen 400 shown in FIG. 4 is used. The head holder selection screen 400 is displayed on the display section 125 and includes a size setting section 401 and a material setting section 402.

The size of the head holder 201 is set in the size setting section 401. As shown in FIG. 4, Middle is selected from three options of Large, Middle, and Small, and the size corresponding to Middle is set in the size setting section 401. The size corresponding to each of the Large, Middle, and Small are stored in the storage section 123 in advance.

The material of the head holder 201 is set in the material setting section 402. As shown in FIG. 4, Soft is selected from Hard and Soft, and a material corresponding to Soft is set in the material setting section 402. The material corresponding to each of Hard and Soft is stored in the storage section 123 in advance together with the X-ray attenuation coefficient of each material.

### S302

The operator sets an X-ray irradiation condition, which is a condition of an X-ray dose with which the subject 10 is irradiated from the X-ray source 101, via the operation unit 120. The X-ray irradiation condition, for example, is set to a constant irradiation dose D_0 at all rotation angles θ as in a dotted line of the graph of FIG. 2, is set such that an irradiation dose to the site having high radiation sensitivity is reduced as compared with an irradiation dose to other sites as in a solid line of the graph of FIG. 2. In addition, the irradiation dose may be set such that the transmission dose at each rotation angle θ falls within a predetermined range. In the following, a case where the X-ray irradiation condition is set as a solid line in the graph of FIG. 2 will be described.

### S303

The system controller 124 calculates a range of projection angle at which the X-ray passes through the head holder 201 or the top plate 202. The projection angle at which the X-ray passes through the head holder 201 or the top plate 202 is a projection angle at which a line connecting the X-ray focus of the X-ray source 101 and the center channel of the X-ray detector 106, which crosses at least one of the head holder 201 or the top plate 202. The range of the projection angle is calculated based on the size of the head holder 201 and the size of the top plate 202 set in S301.

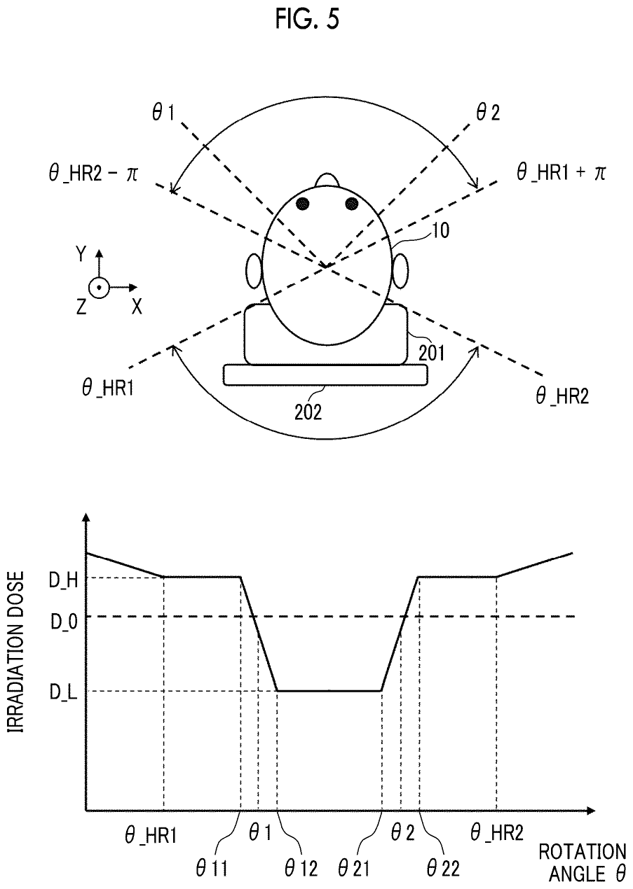

An upper part of FIG. 5 shows an example of a range of projection angle at which the X-ray passes through the head holder 201 or the top plate 202 by a solid line. That is, θ≤θ_HR1, θ_HR2−π≤θ≤Θ_HR1+π, and θ_HR2≤θ are the ranges of the projection angle.

### S304

The system controller 124 changes the X-ray irradiation condition set in S302 such that the irradiation dose from the side on which the head holder 201 or the top plate 202 is disposed is increased according to the X-ray attenuation by the head holder 201 or the top plate 202, in the range of the projection angle calculated in S303.

A lower part of FIG. 5 shows the X-ray irradiation condition in which the irradiation dose is increased according to the X-ray attenuation by the head holder 201 or the like, in θ≤θ_HR1 and θ_HR2≤θ, which are the ranges of the projection angle at which the X-rays irradiated from the side on which the head holder 201 or the top plate 202 is disposed passes through the head holder 201 or the like. The X-ray attenuation in the head holder 201 or the top plate 202 is calculated for each projection angle based on the length of a line connecting the X-ray focus of the X-ray source 101 and the center channel of the X-ray detector 106, which crosses the head holder 201 or the like, and the X-ray attenuation coefficient of the head holder 201 or the like. The changed X-ray irradiation condition is not limited to that in FIG. 5.

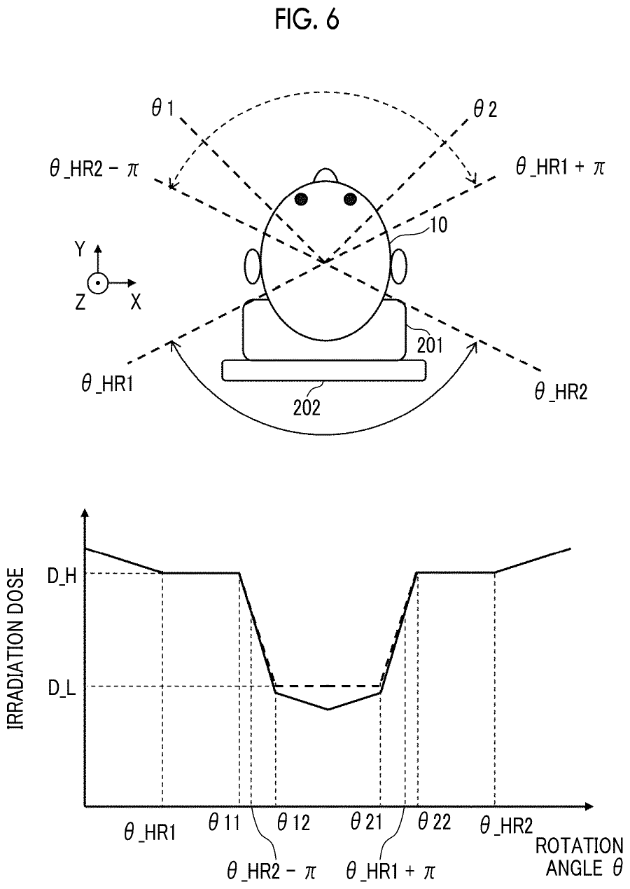

Another example of the changed X-ray irradiation condition will be described with reference to FIG. 6. In a case in which the irradiation dose is increased in a range of the projection angle at which the X-ray irradiated from the side on which the head holder 201 or the like is disposed passes through the head holder 201 or the like, the exposure dose to the subject 10 increases. Therefore, the X-ray irradiation condition is changed such that the irradiation dose from the opposite side is reduced by amount of increase in the irradiation dose from the side on which the head holder 201 or the like is disposed.

An upper part of FIG. 6 shows a range of the projection angle for increasing the irradiation dose by a solid line, and a range of the projection angle for reducing the irradiation dose by a dotted line. Further, a lower part of FIG. 6 shows the X-ray irradiation condition in which the irradiation dose is increased in θ≤θ_HR1 and θ_HR2≤θ, which are the ranges of the projection angle on the side on which the head holder 201 or the like is disposed, and the irradiation dose is reduced in θ_HR2−π≤θ≤θ_HR1+π, which is the range of the projection angle on the opposite side. The lower part of FIG. 6 shows the X-ray irradiation condition shown by the solid line in the lower part of FIG. 5 by the dotted line. Return to the description of FIG. 3.

### S305

The system controller 124 images the subject 10 using the X-ray irradiation condition changed in S304 and generates a tomographic image of the subject 10 using the projection data obtained by the imaging. The generated tomographic image is displayed on the display section 125 or stored in the storage section 123 in order to be used for the image diagnosis of the subject 10.

Since the X-ray irradiation condition is changed to compensate for the X-ray attenuation in the head holder 201 or the top plate 202 by the flow of processing described with reference to FIG. 3, it is possible to suppress the decrease in the image quality of the tomographic image. Further, as shown in FIG. 6, the irradiation dose from the opposite side is reduced by compensating for the X-ray attenuation of the head holder 201, and the exposure dose to the subject 10 can be suppressed.

### Example 2

In Example 1, a case where the X-ray irradiation condition is changed according to the type of the head holder 201 selected by the operator has been described. In Example 2, a case where the X-ray irradiation condition is changed according to the type of the head holder 201 estimated by the camera image or the like will be described.

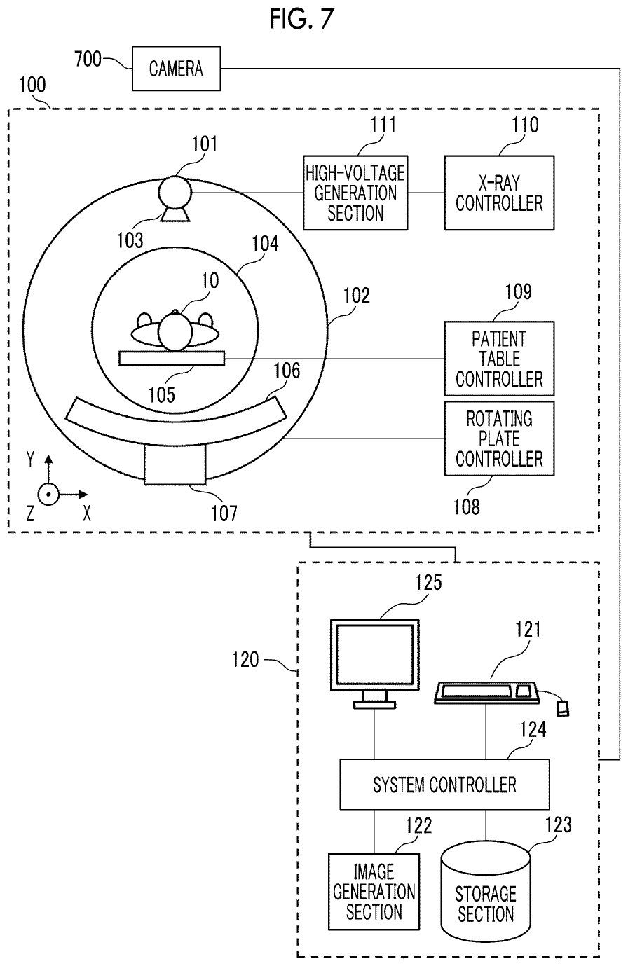

An overall configuration of the X-ray CT apparatus of Example 2 will be described with reference to FIG. 7. The difference from FIG. 1 is that a camera 700 is added, and the description of other components will be omitted.

The camera 700 is a device that images the subject 10 placed on the patient table 105 with the patient table 105 or the head holder 201 from above, and is provided on a ceiling of the imaging room. The camera image captured by the camera 700 is transmitted to the system controller 124 and is used for estimating the size and the material of the head holder 201.

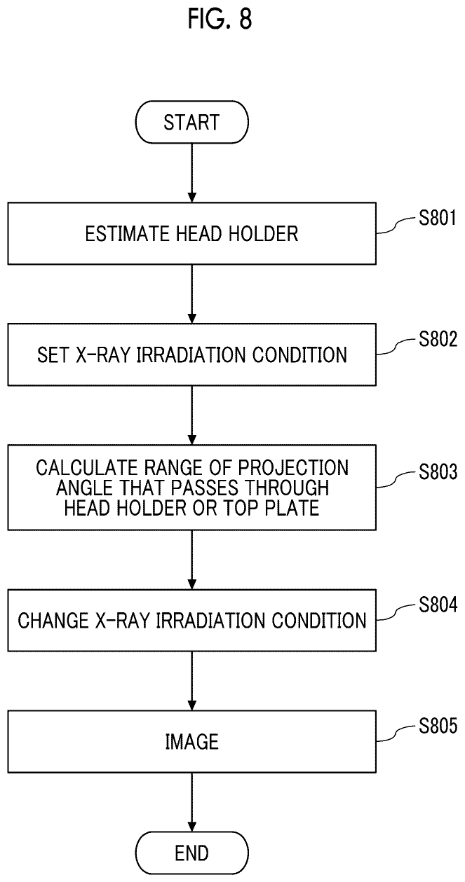

An example of a flow of processing of Example 2 will be described step by step with reference to FIG. 8.

### S801

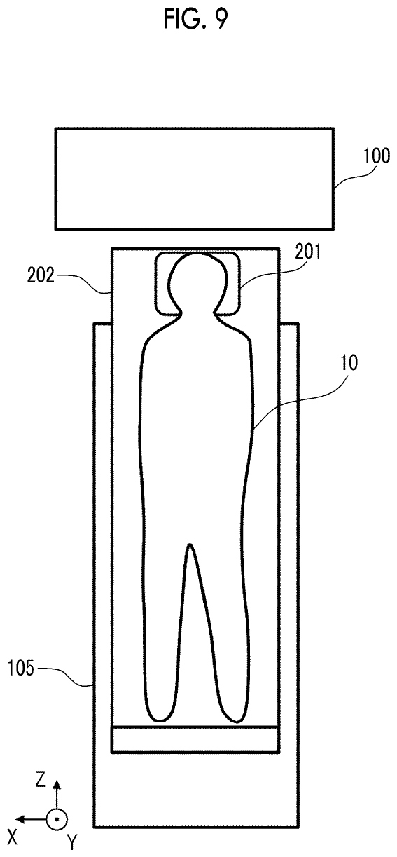

The system controller 124 estimates the size and the material of the head holder 201 based on the camera image captured by the camera 700. For the estimation of the size and the material of the head holder 201, for example, the camera image shown in FIG. 9 is used. Since the shape of the top plate 202 is not uniform in the rotation axis direction of the rotating plate 102, the camera 700 may recognize the size of the top plate 202 that varies depending on the imaging site. In addition, even in a case in which the X-ray shield is present around the subject 10, the X-ray shield may be recognized by the camera 700 in the same manner as the top plate 202. A registration image, which is an X-ray fluoroscopic image acquired before CT imaging, may be used to estimate the size and the material of the head holder 201 instead of the camera image. In addition, artificial intelligence (AI) that is generated in advance by learning a large number of camera images or registration images, and the size and X-ray attenuation coefficient of the head holder 201 as training data may be used for the estimation processing of the head holder 201. The accuracy of the estimation processing can be improved by using the AI.

### S802

The operator sets the X-ray irradiation condition, which is a condition of the X-ray dose with which the subject 10 is irradiated from the X-ray source 101, via the operation unit 120 in the same manner as in S302.

### S803

The system controller 124 calculates a range of projection angle at which the X-ray passes through the head holder 201 or the top plate 202. The range of the projection angle is calculated based on the size of the head holder 201 and the size of the top plate 202 estimated in S801.

### S804

The system controller 124 changes the X-ray irradiation condition set in S802 such that the irradiation dose from the side on which the head holder 201 or the top plate 202 is disposed is increased according to the X-ray attenuation by the head holder 201 or the top plate 202, in the range of the projection angle calculated in S803.

### S805

The system controller 124 images the subject 10 using the X-ray irradiation condition changed in S804 and generates a tomographic image of the subject 10 using the projection data obtained by the imaging. The generated tomographic image is displayed on the display section 125 or stored in the storage section 123 in order to be used for the image diagnosis of the subject 10.

Since the X-ray irradiation condition is changed to compensate for the X-ray attenuation in the head holder 201 or the top plate 202 by the flow of processing described with reference to FIG. 8, it is possible to suppress the decrease in the image quality of the tomographic image. In addition, since the size and the material of the head holder 201 are estimated by the camera image or the like, the burden on the operator is reduced.

The embodiments of the present invention have been described above. The present invention is not limited to the above-described embodiments, and the components can be modified and embodied without departing from the gist of the invention. Additionally, a plurality of components disclosed in the above-described embodiments may be combined as appropriate. Furthermore, some components may be deleted from all the components described in the above-described embodiments.

### EXPLANATION OF REFERENCES

10: subject
100: scan gantry unit
101: X-ray source
102: rotating plate
103: collimator
104: gantry aperture
105: patient table
106: X-ray detector
107: data collection section
108: rotating plate controller
109: patient table controller
110: X-ray controller
111: high-voltage generation section
120: operation unit
121: input section
122: image generation section
123: storage section
124: system controller
125: display section
201: head holder
202: top plate
400: head holder selection screen
401: size setting section
402: material setting section
700: camera

What is claimed is:

1. An X-ray CT apparatus comprising:
an X-ray source that irradiates an X-ray to a subject;
an X-ray detector that detects the X-ray transmitted through the subject;
a rotating plate that causes the X-ray source and the X-ray detector to rotate around the subject;
an image generation section that generates a tomographic image of the subject based on a detection signal of the X-ray detector; and
a controller that controls each section,
wherein the controller changes an irradiation condition of the X-ray irradiated from the X-ray source while the rotating plate rotates, such that an irradiation dose from a side on which a head holder or a top plate is disposed is increased in a range of a projection angle at which the X-ray passes through the head holder or the top plate.

2. The X-ray CT apparatus according to claim 1,
wherein the controller changes the irradiation condition such that an irradiation dose from a side opposite to the side on which the head holder or the top plate is disposed is reduced by an amount of increase in the irradiation dose from the side on which the head holder or the top plate is disposed.

3. The X-ray CT apparatus according to claim 1,
wherein the controller calculates the range of the projection angle at which the X-ray passes through the head holder or the top plate, based on a size of the head holder or a size of the top plate, and
calculates an amount of increase in the irradiation dose from the side on which the head holder or the top plate is disposed, based on a length at which the X-ray crosses the head holder or the top plate and an X-ray attenuation coefficient of the head holder or the top plate.

4. The X-ray CT apparatus according to claim 3,
wherein the size and the X-ray attenuation coefficient of the head holder are set based on selection in a selection screen for selecting a type of the head holder.

5. The X-ray CT apparatus according to claim 3,
wherein the controller estimates the size and the X-ray attenuation coefficient of the head holder based on a camera image or a registration image captured by a camera that images the subject from above.

6. The X-ray CT apparatus according to claim 5,
wherein the controller uses an AI that is generated in advance by learning a large number of the camera images or the registration images, and the size and the X-ray attenuation coefficient of the head holder as training data, in the estimation of the size and the X-ray attenuation coefficient of the head holder.
